(19) United States
(12) Patent Application Publication
(10) Pub. No.: US 2024/0424118 A1
(43) Pub. Date: Dec. 26, 2024

(54) VECTORS TARGETING BETA-D-N-ACETYLGLUCOSAMINIDASE

(71) Applicants: CENTRE NATIONAL DE LA RECHERCHE SCIENTIFIQUE, Paris (FR); UNIVERSITE DE POITIERS, POITIERS CEDEX (FR)

(72) Inventors: Sébastien PAPOT, POITIERS (FR); Brigitte RENOUX, NOUAILLE MAUPERTUIS (FR); Rémi CHÂTRE, ISSOUDUN (FR)

(21) Appl. No.: 18/692,318

(22) PCT Filed: Sep. 13, 2022
(86) PCT No.: PCT/EP2022/075426

§ 371 Date: Mar. 14, 2024

(30) Foreign Application Priority Data
Sep. 14, 2021 (FR) 2109610

(51) Int. Cl. A61K 47/64; A61P 35/00

(52) U.S. Cl. CPC A61K 47/643; A61P 35/00

(57) ABSTRACT

The present invention relates to compounds of formula (I), wherein:

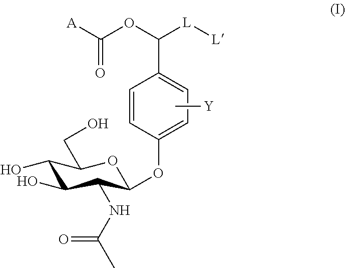

wherein:
   A is an anti-cancer agent,
   Y is an electron-withdrawing or electron-donating group,
   L represents a linker -A₁-A₂-A₃-A₄-A₅-A₆-
   A₁, A₃ and A₅ representing a (C₁-C₆) alkylene radical,
   A₂ representing a group obtained by click chemistry,
   A₄ being a (C₁-C₃₂) alkylene radical interrupted by at least one oxygen atom,
   A₆ being a radical selected from the group consisting of: —NR_{c}—, —O— and —S—, R_{c} representing H or a (C₁-C₁₂) alkyl group, and
   L′ is a radical capable of reacting with an amino, hydroxy or thiol function.

The present invention relates to novel vectors targeting beta-D-N-acetylglucosaminidase, as well as the corresponding prodrugs. It also relates to said vectors and prodrugs for their use for the treatment of cancers.

Cancer and inflammatory diseases are among the most common pathological conditions at the present time. Among the various possible treatment modes, chemotherapy is only usable against circulating tumors, such as lymphomas and leukemias, and metastases. Among the active ingredients that can be envisaged in chemotherapy are certain natural peptide compounds such as, in particular, dolastatin 10, a linear natural compound derived from the marine world, consisting of four amino acids, three of which are specific. Synthetic derivatives of dolastatin 10 are currently also available and preferred. They more particularly include auristatin PE, auristatin E, and monomethyl auristatin E (MMAE). Dolastatin, auristatin E and derivatives thereof possess the property of inhibiting the polymerization of tubulin and thus preventing cell division (antimitotics). However, these active ingredients of the dolastatins family unfortunately lack satisfactory selectivity with respect to tumor cells, just as other anticancer active agents used clinically do. Indeed, they also target healthy tissues. This non-selective destruction causes severe side effects and in most cases leads to premature stopping of the treatment. The development of novel anti-cancer agents capable of selectively destroying tumors without affecting healthy organs is therefore of major interest in combating cancer.

Targeting β-glucuronidase with glucuronylated vectors capable of binding in vivo to albumin is a very effective strategy for treating a large variety of malignant pathologies. Its expression in the tumor microenvironment makes this enzyme a target of choice for the selective release of various active ingredients. On the other hand, with the aim of developing vectorized polychemotherapies, the discovery of other enzymes present in large concentration in the extracellular matrix of solid tumors is of great interest. The activity of β-glucuronidase is in fact reduced in the tumor microenvironment due to the pH which is not optimal for it to operate in (6-6.5). Thus, the saturation of this enzyme, caused by the inflow of a large quantity of vector into the malignant tissues, could constitute a limit for this targeting strategy. In this context, the development of new glycosylated triggers targeting other enzymes than β-glucuronidase is an interesting alternative to overcome this problem. Indeed, the use of a cocktail of substrate vectors of different enzymes should make it possible to limit the saturation of the activity of a single enzyme and thus lead to the selective release of a higher concentration of the anti-cancer agent into the tumor. In addition, this approach could be suitable for targeting different active molecules whose release would be controlled by a specific glycosidase during vectorized polychemotherapies, a strategy still never employed to date.

β-D-N-acetylglucosaminidase is a lysosomal glycosidase present in the vast majority of cells. It has a selective hydrolytic activity of the N-acetylglucosamine residues present on the glycoproteins which contributes to the activation or inactivation of the properties thereof. The presence of this design is derived from an equilibrium between the activity of D-N-acetylglucosaminyltransferase, which fixes the N-acetylglucosamine residues, and that of β-D-N-acetylglucosaminidase. From this equilibrium, numerous signaling processes that are important for cell proliferation will result. An imbalance of this homeostasis was observed in certain cases of cancer, leading to the appearance of oncogenic properties in proteins (A. Peixoto, M. Relvas-Santos, R. Azevedo, L. L. Santos, J. A. Ferreira, Front. Oncol., 2019, 9, 380; H. Nie, H. Ju, J. Fan, X. Shi, Y. Cheng, X. Cang, Z. Zheng, X. Duan, W. Yi, Nat. Commun. 2020, 11, 36; R. Muniz de Queiroz, R. Madan, J. Chien, W. B. Dias, C. Slawson, JBC, 2016, 291, 18897-18914; Z. Ma, K. Vosseller, J B C, 2014, 289, 34457-34465 and J. A. Hanover, W. Chen, M. R. Bond, J. Bioenerg. Biomembr., 2018, 50, 155-173).

Although increasing the concentration of β-N-acetylglucosaminidase is an interesting feature for the selective release of active ingredients, very few substrate compounds of this enzyme have been developed for therapeutic purposes.

The purpose of the present invention is therefore to provide new vectors of the enzyme β-N-acetylglucosaminidase.

Thus, the present invention relates to a compound having the following formula (I):

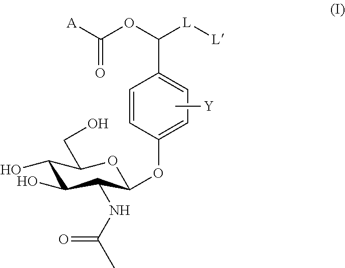

wherein:
   A is an anti-cancer agent,
   Y is an electron-withdrawing or electron-donating group,
   L represents a linker corresponding to the following formula (II):

-A₁-A₂-A₃-A₄-A₅-A₆- (II)
wherein:
   A₁ represents a (C₁-C₆)alkylene radical, in particular —CH₂—,
   A₂ represents a group obtained by click chemistry, in particular triazole,
   A₃ represents a (C₁-C₆)alkylene radical, in particular —CH₂—,
   A₄ represents a (C₁-C₃₂)alkylene radical interrupted by at least one oxygen atom, and preferably being a polyoxyalkylene radical, in particular —(CH₂—O—CH₂)₁₀—,
   A₅ represents a (C₁-C₆)alkylene radical, in particular —CH₂—,
   A₆ is a radical selected from the group consisting of: —NR_{c}—, —O— and —S—, R_{c} representing H or a (C₁-C₁₂)alkyl group, in particular —NH—, and
   L′ represents a radical capable of reacting with an amino, hydroxy or thiol function, and preferably a thiol function,
and pharmaceutically acceptable salts thereof, or a racemic diastereomeric or enantiomeric mixture thereof.

The compounds of formula (I) according to the invention are vectors of the anti-cancer agent A and substrates of the β-N-acetylglucosaminidase.

The compounds of formula (I) can have asymmetric centers. The compounds of the present invention containing an asymmetrically substituted atom can be isolated in optically active or racemic forms. It is well known in the field of the invention how to prepare optically active forms, such as by resolution of racemic forms or by synthesis from optically active starting materials. All the chiral, diastereoisomeric, racemic forms and all the geometric isomers forms of a compound are targeted, unless the stereochemistry or the isomer form is specifically indicated.

The term “pharmaceutically acceptable salt” refers to salts that retain the biological efficacy and the properties of the compounds of the invention and that are not biologically or otherwise undesirable. In many cases, the compounds of the invention are capable of forming acid and/or base salts by virtue of the presence of amino and/or carboxyl groups or of groups similar to these. The pharmaceutically acceptable acid addition salts can be prepared from inorganic and organic acids, while pharmaceutically acceptable base addition salts can be prepared from inorganic and organic bases. For a review of pharmaceutically acceptable salts, see Berge et al. ((1977) J. Pharm. Sd, vol. 66, 1). The expression “pharmaceutically acceptable salts” refers to non-toxic salts formed with non-toxic, pharmaceutically acceptable inorganic or organic acids or inorganic or organic bases. For example, the salts comprise those derived from inorganic acids such as hydrochloric, hydrobromic, sulfuric, sulfamic, phosphoric, nitric and similar acids, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfaniic, fumaric, methanesulfonic, and toluenesulfonic and similar acids.

As mentioned above, A is an anti-cancer agent.

According to one embodiment, the anti-cancer agent is selected from cytostatics, antimetabolites, DNA intercalators, topoisomerase I and II inhibitors, tubulin inhibitors, alkylating agents, neocartinostatin, calicheamycin, dynemicin or spiramycin A, ribosome inhibitors, tyrosine phosphokinase inhibitors, cell differentiation-inducing compounds, histone deacetylase inhibitors, small molecule immunomodulators, or small molecules targeting cancerous stem cells.

Even more particularly, the anti-cancer agent according to the invention is selected from cytostatics and antimetabolites, such as 5-fluorouracil, 5-fluorocytidine, 5-fluorouridine, cytosine arabinoside or methotrexate, from DNA intercalators such as doxorubicin, daunomycin, idarubicin, epirubicin. or mitoxantrone, from topoisomerase 1 and II inhibitors such as camptothecin, etoposide or m-AMSA, from tubulin inhibitors such as vincristine, vinblastine, vindesine, taxol, nocodazole or colchicine, from alkylating agents, such as cyclophosphamide, mitomycin C, rachelmycin, cisplatin, mustard gas phosphoramide, melphalan, bleomycin, N-bis(2-chloroethyl)-4-hydroxyaniline, or neocarzinostatin, calicheamicin, dynemicin or spiramycin A, or from ribosome inhibitors, such as verrucarin A, from tyrosine phosphokinase inhibitors, such as quercetin, genistein, erbstatin, tyrphostin or rohitukin and their derivatives, from compounds that induce cell differentiation, such as retinoic acid, butyric acid, phorbol esters or aclacinomycin, from histone deacetylase inhibitors such as CI-994 or MS275, from immunomodulators such as Imiquimod, and from small molecules targeting cancerous stem cells, such as hedgehog inhibitors like cyclopamine derivatives.

According to one embodiment, the anti-cancer agent according to the invention is chosen from angiogenesis inhibitors analogous to compostatin A, hedgehog pathway inhibitors such as cyclopamine, tyrosine kinase inhibitors, or immunostimulants.

The family of dolastatins represents a class of compounds having a structure of at least 4 amino acids, at least 3 thereof being specific, that is, different from the 20 amino acids most commonly found in nature.

Reference may be made in particular to document WO 2004/010957, which describes compounds in accordance with those suitable for the present invention.

In a particularly preferred embodiment of the invention, A represents a radical derived from dolastatin 10, from auristatin PE, from auristatin E, from monomethyl auristatin E and derivatives thereof, preferably a radical derived from monomethyl auristatin E or a derivative thereof.

The structural difference between dolastatin 10 and the synthetic compounds of the auristatin subfamily particularly lies in the substitution of the amino thiazolephenethyl group in the C-terminal position of dolastatin 10, by a norephedrine unit in the case of auristatin PE, of auristatin E or of monomethyl auristatin.

In the context of the present invention, and in one particularly preferred embodiment, the radical of the dolastatin family is advantageously chosen from monomethyl auristatin E (MMAE) and a derivative thereof.

For the purposes of the invention, a derivative of dolastatin 10, of auristatin PE, of auristatin E or of monomethyl auristatin E has a chemical structure very related to at least one of its active agents and has antimitotic properties attributed to the compounds of the dolastatin family.

Its structural difference(s) may in particular be, for example, a substitution on at least one side chain of at least one of the four amino acids of which it is composed.

This substitution may be carried out so as to contain or represent a linear, cyclic and/or branched alkyl group, an aryl group, a heterocycle or a carbocycle.

This structural difference may also consist of a modification of a dolostatin 10, auristatin PE or auristatin E molecule, for example at the level of its tertiary amine in the N-terminal position, so as to render this function compatible with the establishment of a covalent bond with the linker arm under consideration.

It is part of the general knowledge of those skilled in the art to select the modifications most suitable for these purposes.

According to a preferred embodiment, A is monomethyl auristatin E, doxorubicin or a derivative thereof.

Preferably, A is represented by the following formula (A-1):

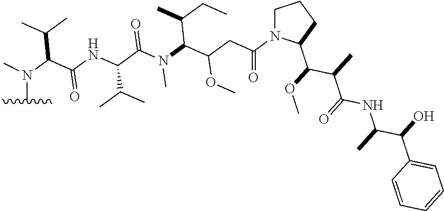 [SMILES: *N(C)[C@H](C(=O)N[C@H](C(=O)N(C)[C@H](C(CC(=O)N1CCC[C@H]1[C@H](OC)[C@@H](C)C(=O)N[C@H](C)[C@@H](O)c1ccccc1)OC)[C@@H](C)CC)C(C)C)C(C)C]

In the context of the present invention, an “electron-withdrawing group” refers to the property of an atom or group of atoms to attract electrons.

Mention may be made, for example, of the groups selected from the group consisting of the group NO₂, esters, CN group, halogen atoms, and alkoxy groups. Preferably, said group is selected from the group consisting of the NO₂ group, the CO₂Me group, the CN group, the fluorine atom, the bromine atom, the iodine atom, the chlorine atom and the methoxy group.

In the context of the present invention, an “electron-donating group” refers to the property of an atom or group of atoms to donate electrons.

An electron-donating group refers is a group selected from the group consisting of the phenyl group, the hydroxy group (OH), a C₁ to C₁₀, preferably C₁ to C₆, linear or branched alkyl, a halogen atom, a hydrogen atom, and an alkoxy group,

Preferably, Y is an electron-withdrawing group, in particular chosen from the group consisting of halogen, NO₂, and CF₃ atoms. Preferably, Y is NO₂.

In the compounds of formula (I) according to the invention, as indicated above, the linker L is such that the group A₆ is connected to the Group L′ and Group A₁ is connected to the carbon atom bearing the group —O—C(═O)-A and the abovementioned phenyl.

According to the present application, the term “alkyl” means a saturated or unsaturated aliphatic hydrocarbon group which can be linear or branched, having, unless otherwise indicated, 1 to 12 carbon atoms in the chain. Preferred alkyl groups have 1 to 6 carbon atoms in the chain. “Branched” means that one or more lower alkyl groups, such as methyl, ethyl or propyl, are attached to a linear alkyl chain. “Lower alkyl” means 1 to 4 carbon atoms in the chain, which may be straight or branched.

The term “alkylene” as used herein refers to a divalent radical comprising, unless otherwise stated, 1 to 6 carbon atoms. An alkylene radical corresponds to an alkyl radical with one hydrogen atom less. Said radical when it is linear can in particular be represented by the formula (CH₂)ₙ wherein n is an integer varying from 1 to 6.

Preferably, in formula (I), A₁ represents a —CH₂— radical.

According to the invention, in formula (I), A₂ is a group obtainable by click chemistry. This radical is thus obtained by a click chemistry reaction.

These click chemistry reactions include in particular the cycloadditions of unsaturated compounds, among which mention may be made of Diels-Alder reactions between a dienophile and a diene, and especially also azide-alkyne 1,3-dipolar cycloadditions, and preferably copper-catalyzed azide-alkyne cycloadditions (CuAAC)

Other click chemistry reactions comprise reactions involving a thiol function such as the formation of thioethers from an alkene and mixed disulphides, as well as reactions involving an electrophilic carbonyl group of the non-aldol type, for example the formation of oxime ethers from an oxyamine, hydrazones from a hydrazine, or the formation of thiosemicarbazones from a thiosemicarbazide.

As click chemistry reactions, mention may also be made of reactions involving thiocarboxylic acids or thioesters to lead to the formation of thioesters and amides, as well as reactions between azides and phosphines (such as Staudinger ligations).

Preferably, the radical A₂ is obtained by reaction between two reactive functions, said reaction being selected from the group consisting of:
the reaction between an azide and an alkyne,
the reaction between an aldehyde or ketone and a hydrazide,
the reaction between an aldehyde or ketone and an oxyamine,
the reaction between an azide and a phosphine,
the reaction between an alkene and a tetrazine,
the reaction between an isonitrile and a tetrazine, and
the reaction between a thiol and an alkene (thiol-ene reaction).

According to a preferred embodiment, A₂ is a triazole group.

Preferably, A₂ is a triazole radical, preferably a radical corresponding to the following formula (II-1):

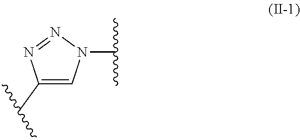 [SMILES: *c1cn(*)nn1]

According to one embodiment, in the aforementioned formula (II), A₃ represents a —CH₂— radical.

According to one embodiment, in the aforementioned formula (II), A₄ represents a polyoxyalkylene radical.

Preferably, A₄ is a group of formula —(CH₂—O—CH₂)ₙ—, n being an integer comprised from 1 to 12. In particular, A₄ is a —(CH₂—O—CH₂)₁₀— group.

According to one embodiment, in the aforementioned formula (II), A₅ represents a —CH₂— radical.

According to one embodiment, in the aforementioned formula (II), A₆ is an —NH-radical.

In formula (I), as mentioned above, L′ is a radical capable of reacting with an amino, hydroxyl or thiol function.

In the context of the present invention, a “radical capable of reacting with an amino, hydroxyl or thiol function” denotes a radical, generally a hydrocarbon radical, which has a function, or chemical unit, capable of interacting with a secondary amino, hydroxyl or thiol function and thus establishing a covalent bond between a conjugated molecule and a distinct chemical entity bearing this function compatible with the production of this covalent function. In the context of the present invention, this distinct chemical entity is more particularly a macromolecule naturally present in a living organism and advantageously a molecule of endogenous albumin, such as human serum albumin.

According to one embodiment, L′ comprises a maleimide radical.

According to one embodiment, L′ conforms to the following formula:

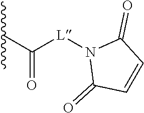

Where L″ represents a (C₁-C₁₂)alkylene radical, optionally substituted by an electron-withdrawing group, in particular a halo(C₁-C₆)alkyl group such as CF₃, or a phenylene radical, optionally substituted with an electron-withdrawing group, in particular a halogen.

According to a preferred embodiment, L′ is a maleimidocaproyl group.

According to one embodiment, the compounds of the invention conform to the following formula (III):

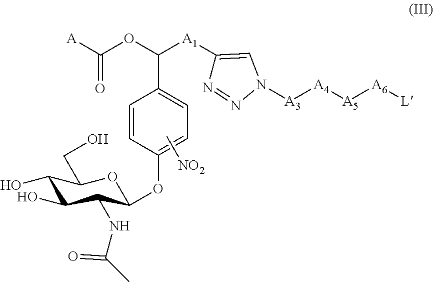

Where A, A₁, A₃, A₄, A₅, A₆ and L′ are as defined above.

According to one preferred embodiment, the compounds of the invention conform to the following formula (IV):

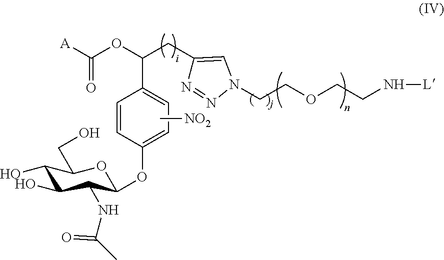

where i is an integer from 1 to 6,
j is an integer from 1 to 6,
n is an integer from 1 to 12, and
A and L′ are as defined above.

A preferred compound according to the invention conforms to the following formula:

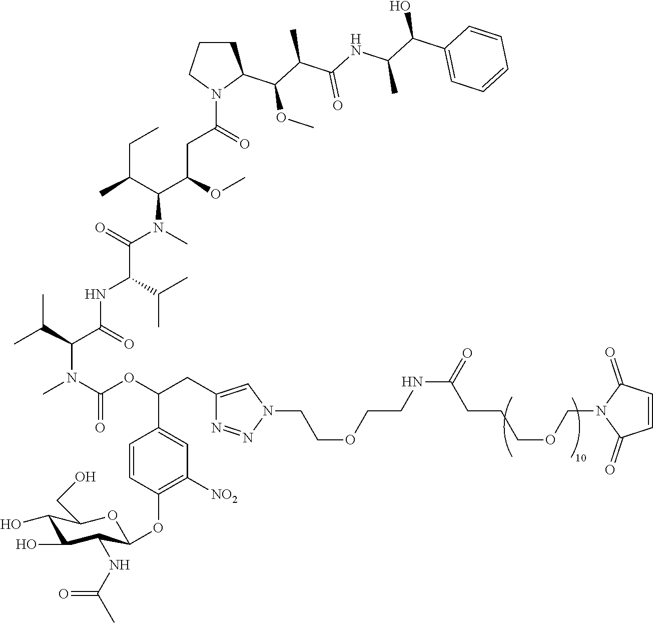

The present invention also relates to a prodrug comprising the compound of formula (I) as defined above linked by a covalent bond to an albumin molecule, or a fragment or derivatives thereof.

Within the meaning of the invention, the term “prodrug” refers to a molecule able to convey in inactivated form an anti-cancer agent, in particular a compound of the dolastatin family, within an organism, and to release that compound into a specifically targeted organ, tissue or cells, under the action of a
β-N-acetylglucosaminidase.

Such a prodrug in particular conforms to the following formula (V):

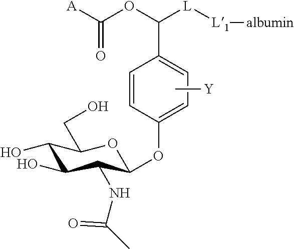

Where A, L and Y are as defined above in formula (I).

The unit L′₁ is for its part derived from the reaction between, on the one hand, the radical L′ comprising a unit capable of reacting with a free amino, hydroxyl or thiol function and in particular with a free thiol function borne by a macromolecule, advantageously an albumin molecule, even more advantageously serum albumin.

In the present application, the prodrug may be formed in vivo or in vitro with a macromolecule, preferably with an albumin molecule.

Thus, an endogenous or exogenous albumin, and in particular human serum albumin, a recombinant albumin or an albumin fragment, may be envisaged.

According to one embodiment, the covalent bond between a molecule of the conjugate, as described by the present invention, and a molecule of endogenous albumin, in particular a human serum albumin molecule, or a derivative thereof, is carried out in vivo.

In one embodiment, a prodrug according to the invention comprises at least one conjugate molecule according to the invention with formula (I) linked by a sulfur thioether bond of the cysteine-34 position of a molecule of endogenous albumin.

It has indeed been shown that a covalent bond is spontaneously established in vivo for example between, on the one hand, a compound bearing a radical capable of reacting with a thiol function and the thiol function of the cysteine-34 position of human albumin (Kratz et al. 2002, J. Med. Chem.).

The invention also relates to a prodrug of the aforementioned formula (V), wherein the group -L′₁-albumin corresponds to the following formula (VI):

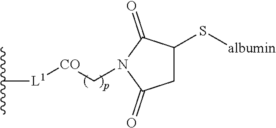

Where L′ is as defined above and p is an integer comprised from 1 to 6, preferably equal to 5.

According to another particular embodiment, a prodrug according to the invention may also be formed in vitro by at least one molecule of the conjugate linked by a covalent bond to an albumin molecule, a recombinant albumin molecule or a fragment of an albumin molecule or a derivative thereof.

Within the meaning of the invention, it is important that the “fragment of an albumin molecule” refers to a fragment of an albumin molecule having a sufficient size to guarantee satisfactory bioavailability, permeability to tumor tissues, and impermeability to the endothelial barrier of healthy tissues, of the prodrug thus generated.

In this particular embodiment, the in vitro coupling between a conjugate of general formula (I), by its radical L′, and an albumin molecule, a recombinant albumin molecule or a fragment of an albumin molecule can be produced with a free and complementary reactive function present at the albumin molecule, the recombinant albumin molecule or the fragment of an albumin molecule.

In a particular embodiment, the fragment of an albumin molecule may comprise the cysteine corresponding to cysteine-34 position of the endogenous albumin sequence.

Contrary to any expectation, the coupling of a conjugate of general formula (I) and of an albumin molecule does not in any way affect the ability of the prodrug thus formed to:
be conveyed and specifically targeted in the microenvironment of the tissue to be treated,
be cleaved into the microenvironment of the tissue to be treated by a β-glucuronidase, and
undergo, after the cleavage of the radical N-acetylglucosaminyl, a rearrangement of the linker arm so as to release the radical representing a compound of the dolastatin family.

In addition, the coupling between a conjugate of general formula (I), by its radical L′, and the amino, hydroxy or thiol function of an albumin molecule, in particular an endogenous one, does not in any way affect the ability of the dolastatin family compound thus released, to exert its biological activity, i.e. its antimitotic activity.

Finally, the coupling between a conjugate of general formula (I), by its radical L′, and the amino, hydroxy or thiol function of an albumin molecule, in particular an endogenous one, limits the removal of the prodrug by the kidneys. The half-life in the blood of a prodrug according to the invention is thus increased in comparison with that of a prodrug represented by a dolastatin family compound functionalized by an N-acetylglucosaminyl radical.

In another embodiment of the invention, the albumin molecule, or albumin fragment, of the prodrug may also be modified, in particular by glycosylation or by PEGylation.

The present invention also relates to a compound or conjugate as defined above with formula (I), or the prodrug as defined above, for its use as a medicament.

The present invention also relates to a pharmaceutical composition comprising a compound as defined above or a prodrug as defined above, or a pharmaceutically acceptable salt, as well as at least one pharmaceutically acceptable excipient.

Although it is possible to administer the compounds of the invention of formula (I) alone, it is preferable to present them in the form of pharmaceutical compositions. The pharmaceutical compositions, both for veterinary and human use, useful according to the present invention comprise at least one compound conforming to formula (I) as defined above, together with one or more pharmaceutically acceptable excipients or carriers and optionally other therapeutic ingredients.

In some preferred embodiments, the active ingredients necessary for the combined therapy can be combined in a single pharmaceutical composition for simultaneous administration.

As used herein, the term “pharmaceutically acceptable” and its grammatical variations, when referring to compositions, supports, diluents and reagents, are used interchangeably, and signify that the materials are capable of being administered to or on a mammal without producing undesirable physiological effects, such as nausea, dizziness, gastric disorders, etc.

The preparation of a pharmacological composition which contains active ingredients dissolved or dispersed therein is well understood in the art and does not need to be limited on the basis of the formulation. Typically, these compositions are prepared in the form of injectable products either in the form of liquid solutions or suspensions; however, solid forms suitable for a solution, or suspensions, in a liquid before use can also be prepared. The preparation can also be emulsified. In particular, the pharmaceutical compositions can be formulated in solid dosage form, for example gel capsules, tablets, pills, powders, tablets or granules.

The choice of the vehicle and the content of active substance in the vehicle are generally determined as a function of the solubility and the chemical properties of the active compound, of the particular administration mode and of the provisions to be observed in pharmaceutical practice. For example, excipients such as lactose, sodium citrate, calcium carbonate, dicalcium phosphate and disintegrant agents such as starch, alginic acids and certain complex silicates associated with lubricants such as magnesium stearate, sodium lauryl sulfate, and talc can be used for the preparation of tablets. To prepare a capsule, it is advantageous to use lactose and high molecular weight polyethylene glycols. When aqueous suspensions are used, they may contain emulsifiers or agents that facilitate the suspension. Diluents such as sucrose, ethanol, polyethylene glycol, propylene glycol, glycerol and chloroform or mixtures thereof can also be used.

The compounds or conjugates of formula (I), the prodrugs or the pharmaceutical compositions according to the present invention can be administered orally, parenterally (subcutaneously, intravenously or intramuscularly) or locally by topical application to the skin and mucous membranes.

The conjugates, prodrugs or pharmaceutical compositions in accordance with the present invention can in particular be administered alone or in combination with chemotherapy or radiation therapy or else in combination, for example, with other therapeutic agents, in particular anti-cancer and anti-mitotic agents, but also in combination with anti-inflammatory agents.

A suitable dosage for the invention can be determined according to a routine approach normally used in the field of the invention. The adjustment of said dosage is clearly part of the general competence of a person skilled in the art.

It is in fact dependent, in particular, on the weight, on the age and on the sex of the individual to be treated, and on the progression of the disease to be treated.

The present invention also relates to the compound of formula (I) according to the invention, or the prodrug as defined above, for the use thereof for the treatment and/or prevention of cancer.

The invention also relates to a method for treating a cancer comprising the administration of a conjugate of general formula (I), of a prodrug as defined above, in particular of general formula (V) or of a pharmaceutical composition, in accordance with the present invention.

The invention also relates to a method for treating a cancer comprising the administration of a conjugate of formula (I), of a prodrug as defined above, in particular of general formula (V) or of a pharmaceutical composition according to the invention, in combination with another treatment selected from a group comprising chemotherapy, radiation therapy, treatment with at least one anti-inflammatory agent, and combinations thereof.

A conjugate or compound of general formula (I), a prodrug according to the invention, in particular of general formula (V), or a pharmaceutical composition according to the present invention may be used, for its use in the prevention and/or treatment of a solid cancer, preferentially chosen from a group comprising neuroblastoma, glioblastoma, osteosarcoma, retinoblastoma, soft tissue sarcoma, central nervous system cancer, nephroblastoma, lung cancer, breast cancer, prostate cancer, colorectal cancer, thyroid cancer, cervical cancer, endometrial cancer, ovarian cancer, kidney cancer, liver cancer, brain cancer, testicular cancer, pancreatic cancer, bone cancer, skin cancer, small bowel cancer, stomach cancer, pleural cancer, esophageal cancer, laryngeal cancer and bladder cancer.

In a particular embodiment, the solid cancer is selected from the group consisting of pancreatic cancer, lung cancer and breast cancer.

In a particular embodiment, a conjugate of general formula (I), a prodrug according to the invention, in particular of general formula (V), or a pharmaceutical composition according to the present invention can be used, for its use in the prevention and/or treatment of metastases.

### FIGURES

FIG. 1 shows the antiproliferative activity of MMAE and of vector 80 (compound according to the invention) in the presence or absence of β-N-acetylglucosaminidase (GlcNase) on the cell lines KB and MBA-MB-231 measured after 72 hours of incubation. The curves with the circles correspond to MMAE, the curves with the squares correspond to vector 80 and the curves with the triangles correspond to vector 80 in the presence of GlcNase.

FIG. 2 shows the therapeutic efficacy of vector 80 for the treatment of MDA-MB-231 orthotopic tumors in mice (initial volume 58 mm³). The vectors were administered on days 21, 35 and 49. Each point corresponds to the average of the tumor volumes±SEM. The curves with the triangles correspond to the vehicle and the curves with the squares correspond to the vector 80.

### EXAMPLES

### Reagents and Solvents

All the reactions were carried out in an argon atmosphere. Unless otherwise indicated, the solvents used were HPLC-grade. The chemical products were of analytical quality from commercial sources and were used without further purification.

### Reaction Monitoring, Purifications, and Analysis

The progress of the reactions was monitored either by liquid chromatography, liquid chromatography coupled with mass spectroscopy or on pre-coated MACHEREY-NAGEL ALUGRAM® SIL G/UV254 silica gel TLC sheets (0.2 mm of silica gel 60). The spots were visualized under UV light at 254 nm and/or by immersing the TLC sheet in a solution of phosphomolybdic acid (3 g) in ethanol (100 mL) followed by heating with a thermal gun.

Automatic chromatographies were carried out with a COMBIFLASH® RF 2001 TELEDYNE ISCO instrument equipped with UV detector and ELSD and using Interchim® 15 or 50 μm silica flash cartridges for normal phase chromatography and HP C18 RediSep® GOLD 4 g or 15.5 g for reverse phase chromatography.

NMR¹H and ¹³C spectra were recorded at 400 MHz and 100 MHz respectively on a Bruker 400 Avance III instrument, equipped with an ultra-shielded magnet and a BBFO 5 mm broadband probe. For the selected compounds, NMR¹H and ¹³C were recorded at 500 MHz and 126 MHz respectively on a Bruker spectrometer equipped with a TXI¹H-¹³C-¹⁵N cryoprobe (5 mm) in the Prism platform from the University of Rennes. Chemical shifts (6) are reported in parts per million (ppm) from low to high field and referenced to the residual solvent. The coupling constants (J) are expressed in hertz (Hz).

The exact mass was determined for all derivatives by their infusion on high-resolution ESI mass spectrometers with CBM/ICA FR2708, at the Université d'Orléans and the Organic Analysis Center of IC2MP at the Université de Poitiers.

The analytical RP-HPLC was carried out on a Dionex Ultimate 3000 system equipped with a variable wavelength UV/Visible detector with a MACHEREY-NAGEL NUCLEOSHELL® reverse phase chromatographic column (150/4.6, RP18, 5 μm) at 30° C. and 1 mL·min⁻¹. Method 1 used a linear gradient composed of A (0.2% TFA in water) and of B (CH₃CN) starting with 20% B and reaching 100% B in 30 min. All the chromatograms were recorded at 254 nm.

The analytical LC-MS was carried out on a Shimadzu LCMS-2020 apparatus. A MACHEREY-NAGEL NUCLEOSHELL® reverse phase chromatographic column (150/4.6, RP18, 5 μm) at 40° C. was used for chromatographic separation at a flow rate of 1 mL·min⁻¹. The effluent from the column was introduced into the electrospray ionization (ESI) source of the mass spectrometer. The analyses were carried out in positive and negative ion mode. The electrospray voltage was set at 4.5 kV. The capillary and heater temperatures were 250° C. and 400° C. respectively. The drying gas (nitrogen) and nebulization gas (nitrogen) flow rates were respectively set at 15 L·min⁻¹ and 1.5·min⁻¹. The data analysis was carried out with the software LabSolutions. The LC/MS experiments were carried out using a linear gradient composed of A (0.1% formic acid in water) and B (0.1% formic acid in CH₃CN) beginning with 20% B and reaching 100% B in 15 min (Method 2).

### Procedures

### Synthesis of Compound 83

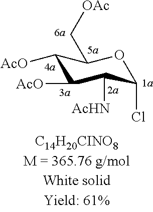

N-acetylglucosamine (3.0 g, 13.5 mmol, 1 equiv.) was solubilized in acetyl chloride (13.5 mL, 190 mmol, 14 equiv.) and the solution was stirred for 72 hours at room temperature. After completion, the solution was hydrolyzed by adding ice-water (150 mL), stirred for 5 minutes and CH₂Cl₂ (60 mL) was added. The organic phase was separated, washed with saturated NaHCO₃ (2×60 mL) and brine (60 mL). The combined organic phases were dried over MgSO₄, filtered and concentrated under vacuum. The crude residue was purified by chromatography on a silica gel column (EP/EtOAc 50/50 to 0/100 in 20 minutes) to give compound 83 (3.0 g, 61%) in the form of a white solid.

R_{f}: 0.37 (EP/EtOAc 40/60)

NMR¹H (400 MHz, CDCl₃, 298K): δ ppm=6.19 (d, J=3.7 Hz, 1H, H₁ₐ), 5.77 (d, J=9.0 Hz, 1H, HNH), 5.32 (dd, J=14.6, 5.5 Hz, 1H), 5.22 (t, J=9.7 Hz, 1H), 4.53 (ddd, J=10.6, 8.8, 3.8 Hz, 1H, H₂ₐ), 4.37-4.23 (m, 2H), 4.14 (dd, J=13.1, 2.7 Hz, 1H), 2.11 (s, 3H), 2.06 (s, 3H), 2.06 (s, 3H), 1.99 (s, 3H).

### Synthesis of Compound 84

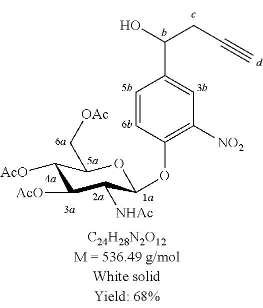

To a solution of phenol 55 (see WO 2011/145068) (169 mg, 0.82 mmol, 1.5 equiv) solubilized in a mixture of CH₂Cl₂ (2 mL) and of aqueous NaHCO₃ (1 M, 1.4 mL), tetrabutylammonium iodide (176 mg, 0.54 mmol, 1 equiv.) was added. The mixture was stirred at room temperature for 15 minutes. Next, a solution of compound 83 (200 mg, 0.54 mmol, 1 equiv.) in CH₂Cl₂ (1 mL) was added and the mixture was stirred until total consumption of the chlorinated compound 83. After 5 h 30, the organic phase was separated, washed with water (5 mL), brine (5 mL), dried over MgSO₄, filtered and concentrated under vacuum. The crude residue was purified by chromatography on a silica gel column (EP/EtOAc 100/0 to 0/100 in 20 minutes) to give compound 84 in the form of a mixture of two diastereoisomers (200 mg, white solid, 68%).

R_{f}: 0.31 (CH₂Cl₂/MeOH 95/5)

NMR¹H (500 MHz, CDCl₃, 298K): 5 ppm=7.85 (2d, J=2.1 Hz, 1H, H_{3b}), 7.62-7.43 (m, 1H, H_{5b}), 7.36 (d, J=8.6 Hz, 1H, H_{6b}), 5.77 (d, J=8.2 Hz, 1H, H_{NH}), 5.59 (dd, J=10.4, 9.1 Hz, 1H, H₃ₐ), 5.50 (dd, J=8.2, 1.1 Hz, 1H, H₁ₐ), 5.13 (t, J=9.5 Hz, 1H, H₄ₐ), 4.97-4.84 (m, 1H, H_{b}), 4.34-4.15 (m, 2H, H₆ₐ), 3.97-3.81 (m, 2H, H₂ₐ and H₅ₐ), 2.73-2.57 (m, 2H, H_{c}), 2.11 (t, J=2.6 Hz, 1H, H_{d}), 2.09 (s, 3H, H_{acetate}), 2.06 (s, 3H, H_{acetate}), 2.05 (s, 3H, H_{acetate}), 1.99 (s, 3H, H_{acetamide}).

NMR¹³C (126 MHz, CDCl₃, 298K): δ ppm=171.28, 170.71, 170.61, 169.63, 148.86, 138.91, 131.29, 122.65, 121.20, 121.14, 99.71, 79.53, 77.41, 77.16, 76.91, 72.37, 72.20, 71.28, 70.83, 68.59, 62.04, 55.48, 29.62, 23.50, 20.91, 20.85, 20.81.

HRMS (ESI): [M+Na]⁺ calculated for C₂₄H₂₈N₂NaO₁₂: 559.1534 found 559.1521.

### Synthesis of Compound 85

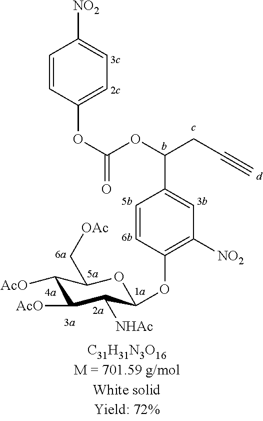

To a phenol solution 84 (200 mg, 0.37 mmol, 1 equiv.) solubilized in CH₂Cl₂ (4 mL), nitrophenyl chloroformate (150 mg, 0.75 mmol, 2 equiv.) was added at room temperature. The mixture was cooled to 0° C. and pyridine (75.2 μl, 0.93 mmol, 2.5 equiv.) was added. The mixture was stirred at 0° C. for 20 minutes and allowed to warm to room temperature for three hours. After completion, the mixture was hydrolyzed with saturated NaHCO₃ (4 mL) and stirred for 5 minutes. Then, the organic phase was separated and the aqueous phase was extracted with CH₂Cl₂ (4 mL). The combined organic phases were washed with brine (5 mL), dried over MgSO₄, filtered and concentrated under vacuum. The crude residue was purified by chromatography on a silica gel column (CH₂Cl₂/MeOH 100/0 to 95/5 in 30 minutes) to give compound 85 in the form of a mixture of two diastereoisomers (188 mg, white solid, 72%).

R_{f}: 0.17 (CH₂Cl₂/MeOH 98/2)

NMR¹H (500 MHz, CDCl₃, 298K): δ ppm=8.28 (d, J=9.2 Hz, 2H, H_{3c}), 7.91 (t, J=2.2 Hz, 1H, H_{3b}), 7.62 (dt, J=8.6, 2.1 Hz, 1H, H_{5b}), 7.43-7.30 (m, 3H, H_{6b} and H_{2c}), 5.91-5.72 (m, 2H, H_{b} and H_{NH}), 5.72-5.54 (m, 2H, H₃ₐ and H₁ₐ), 5.13 (t, J=9.5 Hz, 1H, H₄ₐ), 4.35-4.17 (m, 2H, H₆ₐ), 3.99-3.88 (m, 1H, H₅ₐ), 3.88-3.79 (m, 1H, H₂ₐ), 3.01-2.78 (m, 2H, H_{c}), 2.12-2.08 (m, 4H, H_{d} and H_{acetate}), 2.07 (s, 3H, H_{acetate}), 2.05 (s, 3H, H_{acetate}), 1.98 (s, 3H, H_{acetamide}).

NMR¹³C (126 MHz, CDCl₃, 298K): δ ppm=171.35, 171.31, 170.66, 170.52, 169.61, 155.27, 151.62, 149.92, 145.70, 141.44, 141.30, 133.46, 133.41, 132.46, 132.26, 125.52, 123.76, 123.57, 121.85, 121.83, 120.79, 120.65, 99.31, 99.18, 72.61, 72.41, 71.02, 70.98, 68.54, 61.99, 60.56, 55.69, 55.62, 26.37, 23.50, 21.22, 20.89, 20.83, 20.80, 14.33.

HRMS (ESI): [M+Na]⁺ calculated for C₃₁H₃₁N₃NaO₁₆: 724.1597 found 724.1604.

### Synthesis of Compound 86

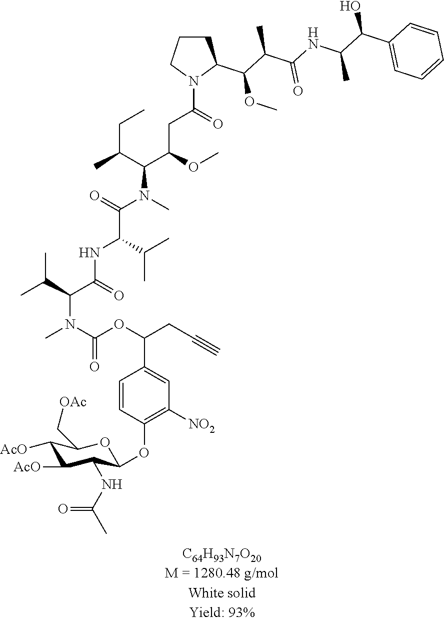

To a carbonate solution 85 (98 mg, 0.139 mmol, 1 equiv.) solubilized in anhydrous DMF (3 mL), MMAE (100 mg, 0.139 mmol, 1 equiv.) and HOBt (19 mg, 0.139 mmol, 1 equiv.) were added. The mixture was stirred at room temperature, and pyridine (0.7 mL) was added. The mixture was stirred at room temperature for 24 hours. After completion, the solvents were evaporated under reduced pressure and the crude residue was purified by chromatography on a column of silica gel (CH₂Cl₂/MeOH 100/0 to 90/10 in 30 minutes) to give the compound 86 in the form of a mixture of 2 diastereoisomers (166 mg, white solid, 93%).

Rt=10.20 min (Method 2)

MS (ESI): [M+H]⁺ calculated for C₆₄H₉₄N₇O₂₀: 1280.6 found 1280.9; [M+2H]²⁺ calculated for C₆₄H₉₅N₇O₂₀: 640.8 found 641.3.

### Synthesis of Compound 63

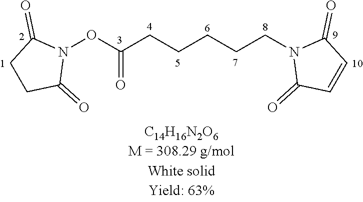 [SMILES: O=C(CCCCCN1C(=O)C=CC1=O)ON1C(=O)CCC1=O]

Maleic anhydride (800 mg, 8.16 mmol, 1 equiv.) was added to a solution of 6-aminohexanoic acid (1.07 g, 8.16 mmol, 1 equiv.) in DMF (10 mL) and stirred at room temperature. After two hours, the mixture was cooled to 0° C., N-hydroxysuccinimide (1.13 g, 9.79 mmol, 1.2 equiv.) and EDC·HCl (3.91 g, 20.4 mmol, 2.5 equiv.) were added, then heated to 35° C. for 12 hours. After cooling to room temperature, the mixture was diluted with CH₂IC₂ (200 mL), washed with NaHCO₃ saturated, water and brine. The organic phase was dried over MgSO₄, filtered and concentrated under reduced pressure. The crude product was purified by chromatography on a silica gel column (EtOAc/EP 60/40) to give compound 63 (1.53 mg, 63%) in the form of a white solid.

Rf: 0.41 (EtOAc/EP 60/40)

NMR¹H (400 MHz, CDCl₃, δ ppm): 6.67 (s, 2H, H₁₀), 3.50 (t, 2H, J=7.17 Hz, H₈), 2.81 (sl, 4H, H₁), 2.58 (t, 2H, J=7.40 Hz, H₄), 1.75 (m, 2H, H₇), 1.61 (m, 2H, H₅), 1.39 (m, 2H, H₆).

NMR¹³C (75 MHz, CDCl₃, δ ppm): 170.8 (C₉), 169.1 (C₂), 168.3 (C₃), 134.0 (C₁₀), 37.4 (C₈), 30.8 (C₄), 28.0 (C₇), 25.8 (C₆), 25.6 (C₁), 24.1 (C₅).

### Synthesis of Compound 80

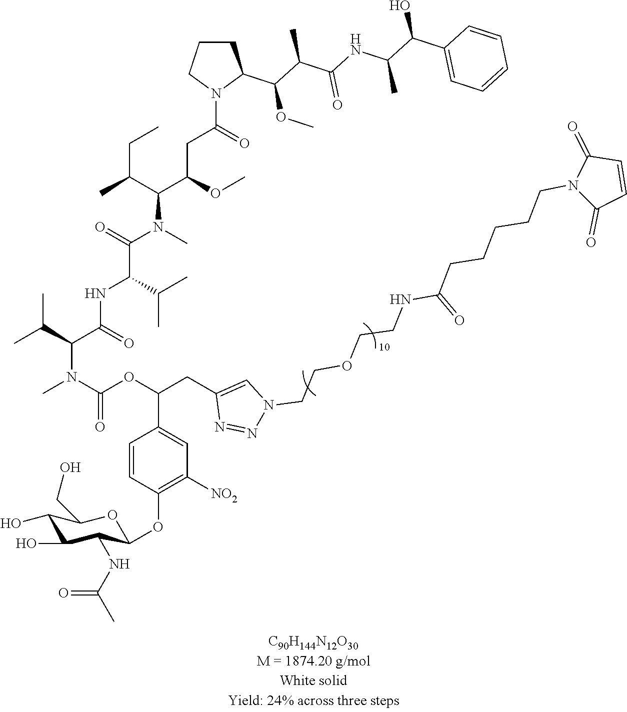

The vector 80 was prepared from the compound 86 in three steps without intermediate purifications.

First, to an alkyne 86 (70 mg, 0.055 mmol, 1 equiv.) and azido-PEG10-amine 46 (32 mg, 0.060 mmol, 1.1 equiv.) solution in degassed CH₂Cl₂ (2 mL) in an argon atmosphere, Cu(MeCN)₄PF₆ (20 mg, 0.055 mmol, 1 equiv.) was added. The mixture was stirred at room temperature until completion. After three hours, the resin QuadraPure® IDA (300 mg) was added to the mixture to trap the copper. The solution was stirred for three additional hours and the resin removed by filtration. The solvent was evaporated under reduced pressure and the crude was used immediately for the next step without further purification.

Second, the crude compound 87 was solubilized in MeOH (2 mL). Next, sodium methanolate (0.44 mg, 0.008 mmol, 0.15 equiv.) was added and the mixture was stirred at room temperature for 5 hours. After completion, the resin IR-120 (300 mg) was added until neutral pH was achieved. The solvent was evaporated under reduced pressure. The crude was used immediately for the next step without further purification.

Finally, triethylamine (23 μL, 0.164 mmol, 3 equiv.) was added to a solution of crude amine 88 and of the ester NHS 63 (18.5 mg, 0.060 mmol, 1.1 equiv.) in anhydrous DMSO (2 mL). The mixture was stirred at room temperature for 1 hour. After completion, controlled by LC-MS (Method 2), the solvent was evaporated under reduced pressure and the crude residue was purified by reverse phase chromatography on C18 grafted silica (elution gradient MeCN/H₂O (0.05% TFA) 20/80 to 80/20 over 30 minutes) to give the vector 80 in the form of a mixture of 2 diastereoisomers (25 mg, white solid, 24% across three steps).

Rt=7.33 min (Method 2)

HRMS (ESI): [M+Na]⁺ calculated for C₉₀H₁₄₄N₁₂NaO₃₀: 1896.0004 found 1895.9952.

### Biological Evaluation of Vector 80

### Evaluation of the Antiproliferative Activity of Vector 80

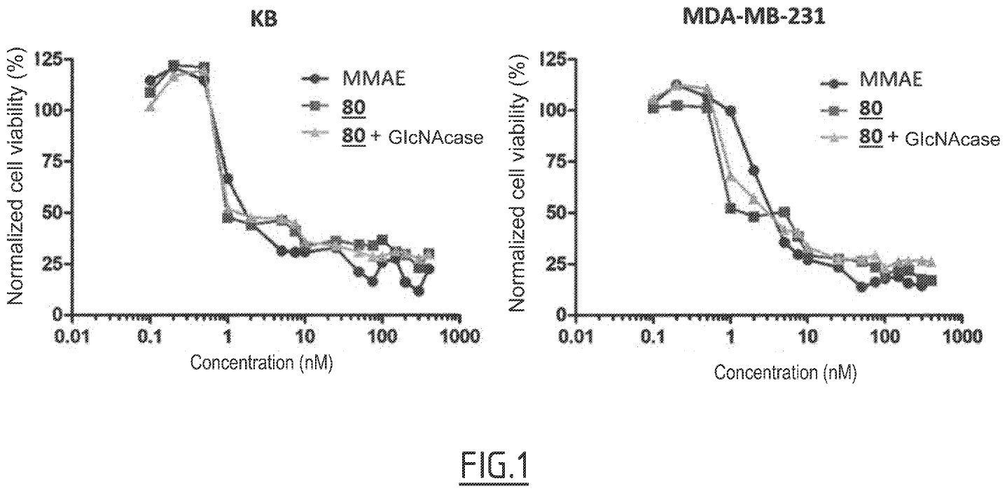

The antiproliferative activity of vector 80 was evaluated on human KB and MDA-MB-231 tumor lines and compared to that of MMAE. To do this, compound 80 was placed in the culture medium in the absence or presence of β-N-acetylglucosaminidase and cell viability was measured after 72 hours of incubation (FIG. 1).

It is noted that vector 80 has an anti-proliferative activity similar to that of MMAE. Indeed, its incubation in the absence or presence of β-N-acetylglucosaminidase leads to identical cytotoxicity, on both cell lines.

This surprising result shows that vector 80 does not make it possible to mask the toxicity of MMAE, contrary to its glucuronylated analog 24. This result could be explained by the difference in polarity between the glucuronides and the N-acetylglucosaminide which do not have ionizable chemical functions (carboxylic acid). Thus, compound 80 could passively penetrate through the cell membrane, then be activated by lysosomal β-N-acetylglucosaminidase to lead to the release of MMAE. A second hypothesis is based on the release of β-N-acetylglucosaminidase by these cancer cells. Indeed, it has been demonstrated that these cancer cells could be secreting this enzyme in the culture medium. This phenomenon could then be responsible for the activation of vector 80 independently of the addition of the β-N-acetylglucosaminidase to the culture medium.

### Evaluation of the Therapeutic Efficacy of the Prodrug 80 In Vivo

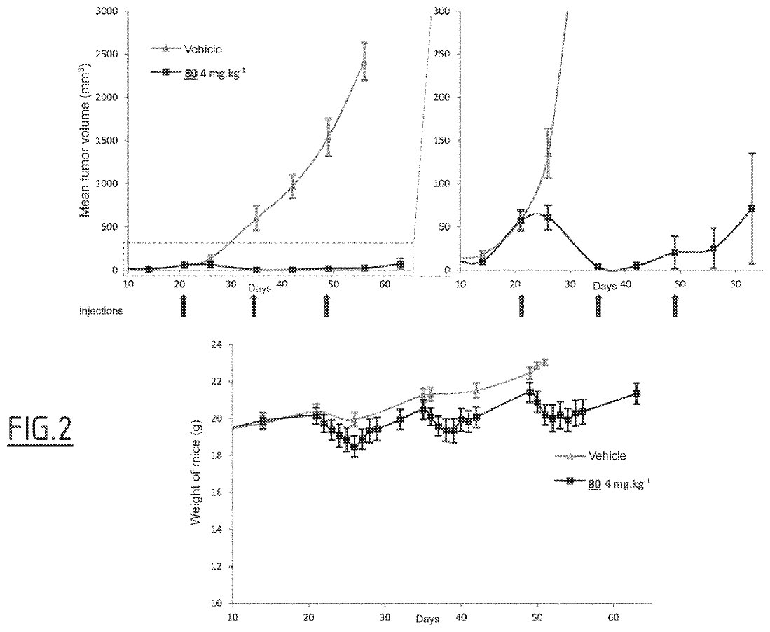

The therapeutic efficacy of vector 80 was evaluated in mice, on a triple negative breast tumor model of the MDA-MB-231 type (FIG. 2). Compound 80 was administered three times, with a period of 14 days, intravenously in the caudal vein, at a dose of 4 mg·kg⁻¹.

It can be seen that vector 80 has significant therapeutic activity relative to the control group. Indeed, a reduction in tumor mass is obtained, as early as the first administration. Furthermore, at the end of the first injection, 4/6 mice treated with vector 80 no longer had detectable tumors from day 35.

Vector 80 was also tolerated well by the animals, since no mortality was observed until the end of the protocol (day 63). At day 56, the treatments with vector 80 led to a strong inhibition of tumor growth relative to the control group (99%). At the end of the experiment (day 63), total and lasting regression of the tumor was obtained for 3/6 mice. On the other hand, a resumption of tumorigenesis was observed for one of the mice which had no longer had a detectable tumor on day 35.

The initial response to treatment with 80 is also very important, leading to a 90% reduction in the initial tumor size. However, at day 42 of the protocol, a resumption of tumor growth was observed. It would also seem that the following administration (day 49) has relatively little effect.

### CONCLUSION

The goal was to study the targeting of β-D-N-acetylglucosaminidase in order to selectively release a cytotoxic agent at the tumors. In this context, the synthesis and the biological evaluation of the vector N-acetylglucosaminylated of MMAE 80 were carried out. The latter comprises a N-acetylglucosamine linked to the MMAE by a self-immolative spacer. The targeting of the cancer tissues is made possible by the presence of the maleimide group of 80 which can react to in vivo with the thiol function of the plasma albumin by a Michael addition reaction.

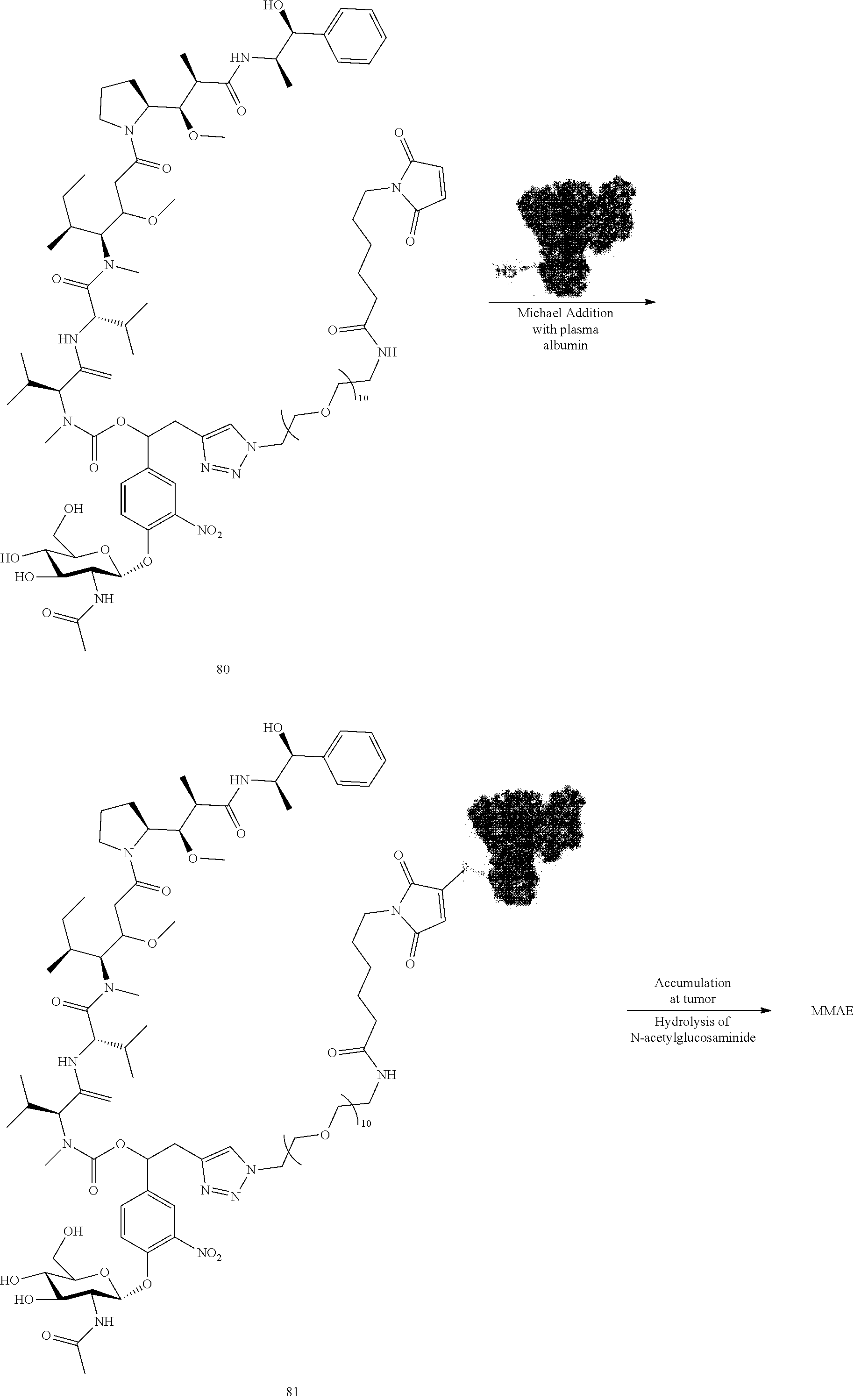

The macromolecular vector 81 formed is then capable of accumulating on the tumor thanks to the physiopathological tropism of the albumin at the malignancy as well as the defects of vascularization linked to tumor neo-angiogenesis by the EPR effect.

The therapeutic efficacy of this new vector was evaluated on a triple negative breast tumor model MBA-MB-231 implanted in mice. These tests showed that vector 80 has a very significant anticancer activity. Thus, the development of N-acetylglucosaminylated vectors is a very promising approach for the development of novel selective and effective treatments of malignant pathologies.

1. A compound of the following formula (I): 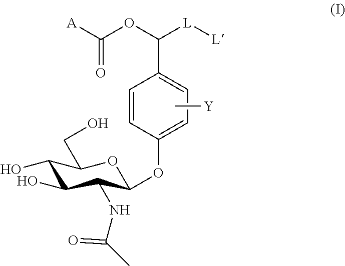
wherein:
A is an anti-cancer agent,
Y is an electron-withdrawing or electron-donating group,
L represents a linker corresponding to the following formula (II):
-A₁-A₂-A₃-A₄-A₅-A₆- (II)
wherein:
A₁ represents a (C₁-C₆)alkylene radical,
A₂ represents a group obtained by click chemistry,
A₃ represents a (C₁-C₆)alkylene radical,
A₄ represents a (C₁-C₃₂)alkylene radical interrupted by at least one oxygen atom, and preferably being a polyoxyalkylene radical,
A₅ represents a (C₁-C₆)alkylene radical,
A₆ is a radical selected from the group consisting of: —NR_{c}—, —O— and —S—, R_{c} representing H or a (C₁-C₁₂)alkyl group, and
L′ represents a radical capable of reacting with an amino, hydroxy or thiol function, and preferably a thiol function,
and pharmaceutically acceptable salts thereof, or a racemic diastereomeric or enantiomeric mixture thereof.

2. The compound of claim 1, wherein A₂ is a triazole group.

3. The compound according to claim 1, having the following formula (III): 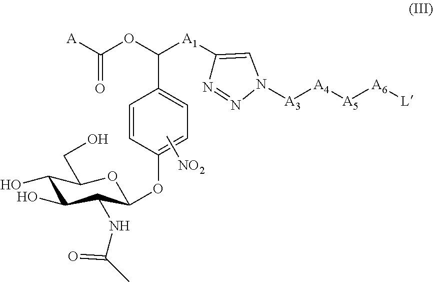
Where A, A₁, A₃, A₄, A₅, A₆ and L′ are as defined in claim 1.

4. The compound according to claim 1, wherein A₄ is a group of formula —(CH₂—O—CH₂)ₙ—, n being an integer comprised from 1 to 12.

5. The compound according to claim 1, such that it conforms to the following formula (IV): 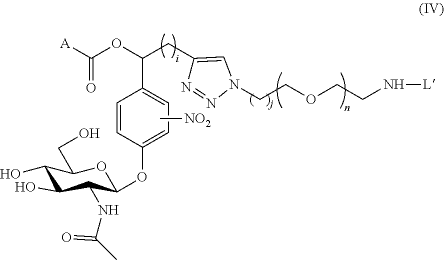
where i is an integer from 1 to 6,
j is an integer from 1 to 6,
n is an integer from 1 to 12, and
A and L′ are as defined in claim 1.

6. The compound according to claim 1, wherein L′ is a maleimidocaproyl group.

7. The compound according to claim 1, wherein A is monomethyl auristatin E, doxorubicin or a derivative thereof.

8. The compound according to claim 1, conforming to the following formula:

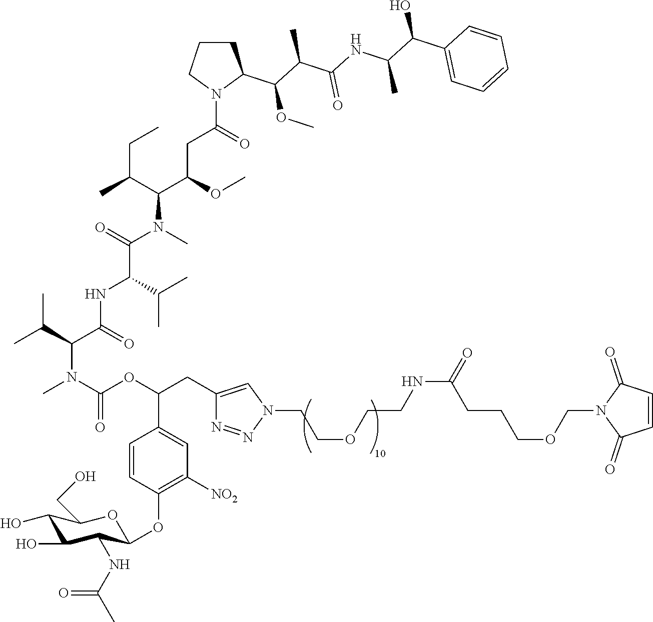

9. A prodrug comprising the compound according to claim 1 bound by a covalent bond to an albumin molecule or a fragment or derivatives thereof.

10. The compound according to claim 1, for use as a medicament.

11. A pharmaceutical composition comprising a compound according to claim 1, or a pharmaceutically acceptable salt, as well as at least one pharmaceutically acceptable excipient.

12. The compound according to claim 1, for use in treating and/or preventing cancer.
